(19) United States
(12) Patent Application Publication
(10) Pub. No.: US 2026/0308123 A1
(43) Pub. Date: Oct. 8, 2026

(54) PROCESS FOR THE PREPARATION OF 2-(PYRID-2-YLOXYMETHYL) PHENYLACETATES

(71) Applicant: CORTEVA AGRISCIENCE LLC, INDIANAPOLIS, IN (US)

(72) Inventors: JAMES J. HENKELIS, PENNINGTON, NJ (US); NICHOLAS M. IRVINE, WESTFIELD, IN (US); MARK ONDARI, FREELAND, MI (US); RAFAEL SHAPIRO, WILMINGTON, DE (US); ERIC WIENSCH, ZIONSVILLE, IN (US)

(73) Assignee: CORTEVA AGRISCIENCE LLC, INDIANAPOLIS, IN (US)

(21) Appl. No.: 19/473,904

(22) PCT Filed: Apr. 10, 2024
(86) PCT No.: PCT/US24/23849

§ 371 Date: Oct. 9, 2025

Related U.S. Application Data
(60) Provisional application No. 63/496,177, filed on Apr. 14, 2023.

(51) Int. Cl. C07D 213/64

(52) U.S. Cl. CPC C07D 213/64

(57) ABSTRACT

Described herein are processes for the preparation of 2-(pyrid-2-yloxymethyl)-phenylacetates which are useful as intermediates for agricultural products. The processes comprise reacting a substituted pyridine with phenylacetic acid alcohol dianions derived from 3-isochromanone. The intermediates may be further reacted to form agricultural products such as fungicides.

### CROSS REFERENCE TO RELATED APPLICATION

This application claims priority to U.S. Patent Application Ser. No. 63/496,177 filed Apr. 14, 2023, and to International Application published under the Patent Cooperation Treaty PCT/US/2024/023849, filed on Apr. 10, 2024, the contents of which are hereby incorporated herein by reference.

### BACKGROUND

Compounds of formula (I) are useful as agricultural chemicals, useful for the control of pests and diseases.

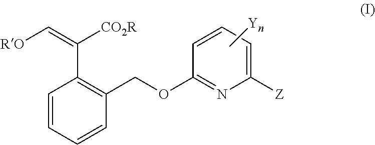

One such compound, picoxystrobin, (E)-Methyl 3-methoxy-2-(2-(((6-(trifluoromethyl)pyridin-2-yl)oxy)methyl)phenyl)acrylate, compound (1), is shown below.

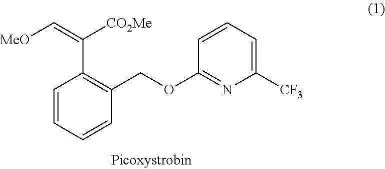 [SMILES: CO/C=C(/C(=O)OC)c1ccccc1COc1cccc(C(F)(F)F)n1]

Picoxystrobin is a broad-spectrum fungicide used against a variety of diseases in wheat, barley, and rye.

General strategies for synthesizing picoxystrobin include treating methyl 2-(6-trifluoromethyl-2-yloxymethyl)phenyl acetate (2) with methyl formate followed by methylation of the enol ether intermediate with dimethyl sulfate (DMS). The same chemistry may be employed in the synthesis of related strobilurin compounds. Other suitable procedures are described in US2004/0152894.

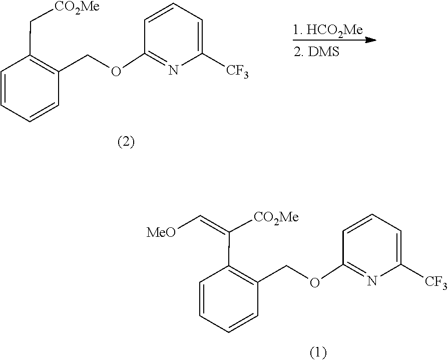 [SMILES: COC(=O)Cc1ccccc1COc1cccc(C(F)(F)F)n1 ; CO/C=C(/C(=O)OC)c1ccccc1COc1cccc(C(F)(F)F)n1]

Routes for the synthesis of compound (2) are discussed in U.S. Pat. Nos. 5,942,623 and 6,015,905. According to U.S. Pat. No. 5,942,623, a disodium salt of 2-hydroxymethylphenylacetic acid (compound 3, M=Na) was reacted with 2-chloro-6-trifluoromethylpyridine (compound 4, L=Cl) in N-methylpyrrolidone. Upon completion of the reaction dimethyl sulfate was added to form the product (2). Other metals M and leaving groups L may be employed in its process.

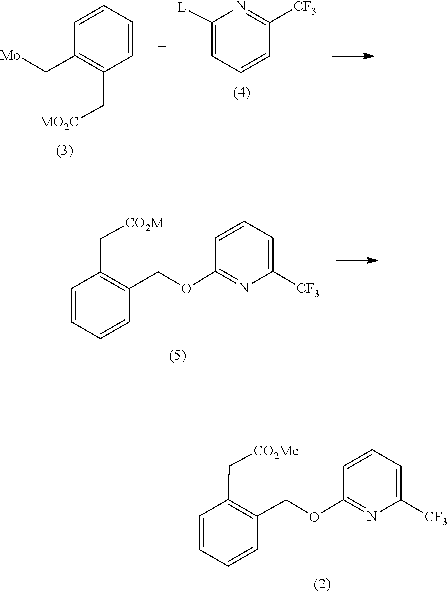

According to U.S. Pat. No. 6,015,905, a sodium salt of 2-hydroxy-6-trifluoromethylpyridine (compound 7, M=Na) was reacted with methyl 2-chloromethylphenylacetate (compound 6, L=Cl). Other metals (M) and leaving groups (L) may be employed in its reaction.

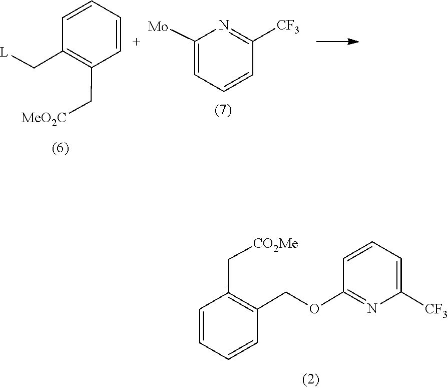

A variety of conditions were tested, and reported yields ranged from 51% to 97%.

Another approach to synthesizing picoxystrobin involves the reaction of enol-ether compound (8) with a metal salt of 2-hydroxy-6-trifluoromethylpyridine (7), where M is Na or K.

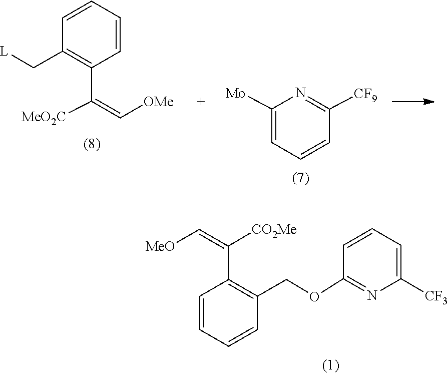

Despite the large amount of work devoted to the synthesis of picoxystrobin and related strobilurin compounds, there remains a continuing need for improved syntheses that address problems of cost, waste, and stereochemical control of the enol-ether double bond.

### DESCRIPTION

Surprisingly, it has been found that reacting a 2-fluoropyridine with the dilithium, disodium, or dipotassium salt of 3-isochromanone followed by esterification results in an unexpectedly high yield of compounds of formula (II),

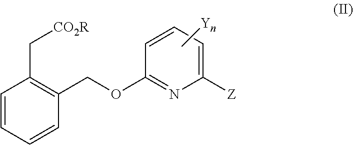

which may then be converted to useful agricultural chemicals, such as compounds of formula (I). A useful compound of formula (I) is (E)-Methyl 3-methoxy-2-(2-(((6-(trifluoromethyl)pyridin-2-yl)oxy)methyl)phenyl)acrylate. In particular, methods described herein provide a highly efficient route to (E)-Methyl 3-methoxy-2-(2-(((6-(trifluoromethyl)pyridin-2-yl)oxy)methyl)phenyl)acrylate.

As described herein, compounds R-L and R′-L′ represent carbon-containing groups R and R′ attached to leaving groups L and L′. A carbon-containing group R or R′ is C₁ to C₄, branched or unbranched, alkyl, alkenyl, alkynyl, haloalkyl, haloalkenyl, or haloalkynyl. Preferred R groups are C₁ to C₄ alkyl, branched or unbranched. A leaving group L or L′ may be halogen as in methyl iodide or methyl bromide or methyl chloride or methyl fluoride; sulfate as in dimethyl sulfate or other dialkyl sulfate; halosulfonate as in methyl fluorosulfonate or methyl chlorosulfonate other alkyl halosulfonate; alkylsulfonate as in methyl methanesulfonate or other alkyl alkylsulfonate; haloalkylsulfonate as in methyl trifluoromethanesulfonate or other alkyl haloalkylsulfonate; phenylsulfonate or substituted-phenylsulfonate as in methyl benzenesulfonate, methyl p-toluenesulfonate, methyl p-brominesulfonate or other alkyl phenylsulfonate or alkyl substituted-phenylsulfonate.

As used herein, a metal suitable for making alkoxides and carboxylates is a metal such as lithium, sodium, potassium, rubidium, cesium, beryllium, magnesium, calcium, strontium, barium, copper, zinc, silver, cadmium, gold, mercury, titanium, or other alkali, or alkaline earth, or transition metal.

As described herein, all reactions involving water-sensitive or oxygen-sensitive reagents are performed under a dry nitrogen atmosphere. All solvents involving water-sensitive or oxygen-sensitive reagents are anhydrous.

As used herein, ranges such as temperature ranges and time-period ranges include the starting and ending points. For example, a temperature range of from 0° C. to 10° C. includes the temperatures of 0° C. and 10° C. Likewise, a time-period of from 1 to 4 hours includes the time periods of 1 hour and four hours.

As used herein, “THF” is tetrahydrofuran, “DMSO” is dimethyl sulfoxide, “DMF” is dimethylformamide, “DMS” is dimethyl sulfate, “FA” is formic acid, “MTBE” is methyl tertiary-butyl ether.

As used herein, a “solution” refers to a compound or multiple compounds mixed with a solvent, or a compound or multiple compounds mixed with a liquid reagent such as methyl formate.

As used herein, a “phase transfer catalyst” (PTC) may be a crown ether, azacrown, thiacrown, phosphonium salt, or a quaternary ammonium salt. Exemplary quaternary ammonium salts include benzyltrimethyl ammonium chloride, benzyltrimethyl ammonium bromide, benzyltrimethyl ammonium hydroxide, benzyltrimethyl ammonium hydrosulfide, benzyltriethyl ammonium chloride, benzyltriethyl ammonium bromide, benzyltriethyl ammonium hydroxide, benzyltriethyl ammonium hydrosulfide, benzyltripropyl ammonium chloride, benzyltripropyl ammonium bromide, benzyltripropyl ammonium hydroxide, benzyltripropyl ammonium hydrosulfide, benzyltributyl ammonium chloride, benzyltributyl ammonium bromide, benzyltributyl ammonium hydroxide, benzyltributyl ammonium hydrosulfide, tetrabutyl ammonium chloride, tetrabutyl ammonium bromide, tetrabutyl ammonium hydroxide, tetrabutyl ammonium hydrosulfide, trioctylmethyl ammonium chloride, trioctylmethyl ammonium bromide, trioctylmethyl ammonium hydroxide, and trioctylmethyl ammonium hydroxide. Preferred quaternary ammonium salts include tetrabutyl ammonium chloride, tetrabutyl ammonium hydrogen sulfate, tetrabutyl ammonium bromide, tetrabutyl ammonium hydroxide and other tetrabutyl ammonium salts.

As used herein, a “one-pot synthesis”, or “one-pot reaction sequence”, or “one-pot method” is a method in which all the steps of the process are performed in a single reaction vessel without removing and purifying intermediate compounds of the process. Compared to multi-pot syntheses, one-pot syntheses may save time and resources while increasing final product yield.

As used herein, “continuous-flow methods or systems”, refers to methods and systems wherein the steps of a process or reaction sequence are performed in an apparatus through which solvent and reactants flow continuously while the individual steps of the process or method are executed. Multi-step continuous-flow systems act as several reactors connected into a single sequential flow. Generally, reagents are pumped into such systems at appropriate locations to execute the desired reactions.

Exemplary methods for preparing compounds of formula (II)

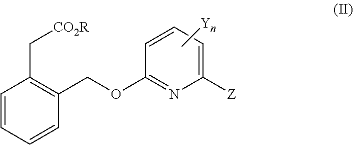

wherein Z is halo, haloalkyl, haloalkoxy, phenyl, phenoxy, amino, alkylamino, dialkylamino, acylamino, cyano, carboxy, alkoxycarbonyl, or alkylcarbonyloxy; and Yₙ wherein n=0, 1, 2, or 3 is independently halo, haloalkyl, haloalkoxy, phenyl, phenoxy, amino, alkylamino, dialkylamino, acylamino, cyano, carboxy, alkoxycarbonyl, or alkylcarbonyloxy; and R is C₁ to C₄, branched or unbranched, alkyl, alkenyl, alkynyl, haloalkyl, haloalkenyl, or haloalkynyl; may comprise:
a) reacting a 2-fluoropyridine of formula (IV) in a solvent;

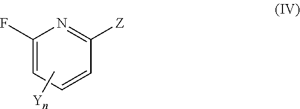

with a compound of formula (III) wherein M is lithium, sodium, potassium, or any other metal suitable for making alkoxides and carboxylates;

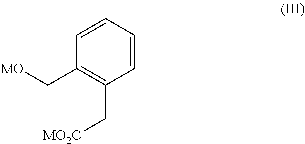

to obtain a compound of formula (V); and

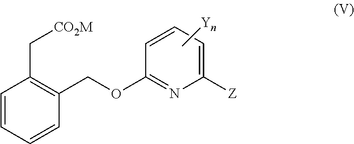

b) reacting (V) with a compound of formula R-L, wherein R is C₁ to C₄, branched or unbranched, alkyl, alkenyl, alkynyl, haloalkyl, haloalkenyl, or haloalkynyl; and L is fluoro, chloro, bromo, iodo, sulfate, fluorosulfonate, alkylsulfonate, phenylsulfonate, substituted phenylsulfonate or the equivalent; to obtain a compound of formula (II).

An exemplary method for preparing a compound of formula (II) may comprise n=0, Z=CF₃, and R=CH₃.

An exemplary method for preparing a compound of formula (II) may comprise M=Na and R-L=dimethyl sulfate.

An exemplary method for preparing a compound of formula (II) may comprise the steps being performed with a solvent selected from the group consisting of water, acetonitrile, DMF, THF, toluene, and mixtures thereof.

An exemplary method for preparing a compound of formula (II) may comprise the use of the solvent toluene.

An exemplary method for preparing a compound of formula (II) may comprise 3-isochromanone reacting with sodium hydroxide in a solvent to produce a compound of formula (III M=Na) to which is then added a solution of 2-fluoro-6-trifluoromethylpyridine.

An exemplary method for preparing a compound of formula (II) may be employed wherein a solution of 3-isochromanone and 2-trifluoro-6-trifluoromethylpyridine are added to sodium hydroxide in a solvent.

An exemplary method for preparing a compound of formula (II) may comprise the steps being performed in one pot.

An exemplary method for preparing a compound of formula (II) may comprise the steps being performed in a continuous flow system.

An exemplary method of preparing a compound of structure (I) wherein Z is halo, haloalkyl, haloalkoxy, phenyl, phenoxy, amino, alkylamino, dialkylamino, acylamino, cyano, carboxy, alkoxycarbonyl, or alkylcarbonyloxy; and wherein Yₙ is independently halo, haloalkyl, haloalkoxy, phenyl, phenoxy, amino, alkylamino, dialkylamino, acylamino, cyano, carboxy, alkoxycarbonyl, or alkylcarbonyloxy and n=0 to 3; and wherein R and R′ are C₁ to C₄, branched or unbranched, alkyl, alkenyl, alkynyl, haloalkyl, haloalkenyl, or haloalkynyl; may comprise:

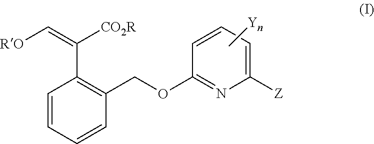

a) reacting a 2-fluoropyridine of formula (IV) in a solvent;

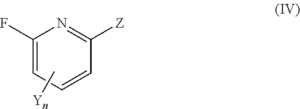

with a compound of formula (III) wherein M is lithium, sodium, potassium, or any other metal suitable for making alkoxides and carboxylates;

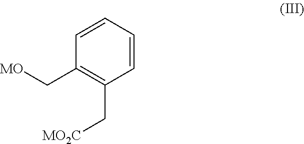

to obtain a compound of formula (V); and

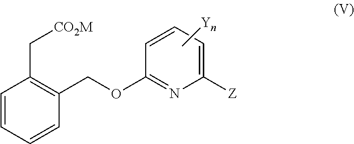

b) alkylating a compound of formula (V) with a compound of formula R-L, wherein L is fluoro, chloro, bromo, iodo, sulfate, fluorosulfonate, alkylsulfonate, phenylsulfonate, substituted phenylsulfonate or the equivalent; to obtain a compound of formula (II); and

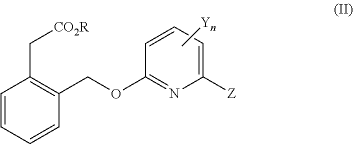

c) reacting a compound of formula (II) with an alkyl formate to form a mixture of compounds of formula (IX); and

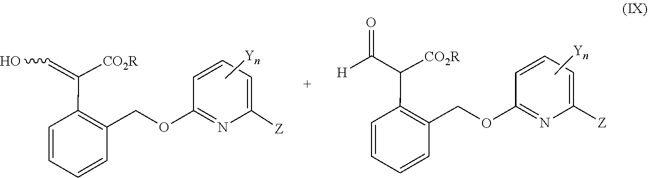

d) alkylating the mixture of compounds of formula (IX) with a compound of formula R′-L′, wherein L′ is fluoro, chloro, bromo, iodo, sulfate, fluorosulfonate, alkylsulfonate, phenylsulfonate, substituted phenylsulfonate or the equivalent; to form a compound of formula (I).

An exemplary method for the synthesis of a compound of formula (I) may comprise n=0, Z=CF₃, R=CH₃, and R′=CH₃.

An exemplary method for the synthesis of a compound of formula (I) may comprise M=Na, R′-L′=dimethyl sulfate, and R-L=dimethyl sulfate.

An exemplary method for the synthesis of a compound of formula (I) may comprise the steps being performed with a solvent selected from the group consisting of water, acetonitrile, DMF, THE, toluene, and mixtures thereof.

An exemplary method for the synthesis of a compound of formula (I) may comprise the solvent toluene.

An exemplary method for the synthesis of a compound of formula (I) may comprise 3-isochromanone being reacted with sodium hydroxide in a solvent to produce a compound of formula (III) to which is added a solution of 2-fluoro-6-trifluoromethylpyridine.

An exemplary method for the synthesis of a compound of formula may comprise a solution of 3-isochromanone and 2-trifluoro-6-trifluoromethylpyridine being added to sodium hydroxide in a solvent.

An exemplary method for the synthesis of a compound of formula (I) may comprise the steps being performed in one pot.

An exemplary method for the synthesis of a compound of formula (I) may comprise the steps being performed in a continuous flow system.

An exemplary method may comprise a final methylation reaction to form a compound of formula (I) employing a phase transfer catalyst.

An exemplary method may comprise the use of a phase transfer catalyst selected from the group consisting of tetrabutylammonium hydroxide, tetrabutylammonium hydrogen sulfate, tetrabutylammonium chloride, and tetrabutylammonium bromide.

An exemplary method may comprise the final product being purified by recrystallization.

An exemplary reaction sequence may begin by reacting 3-isochromanone with alkaline base.

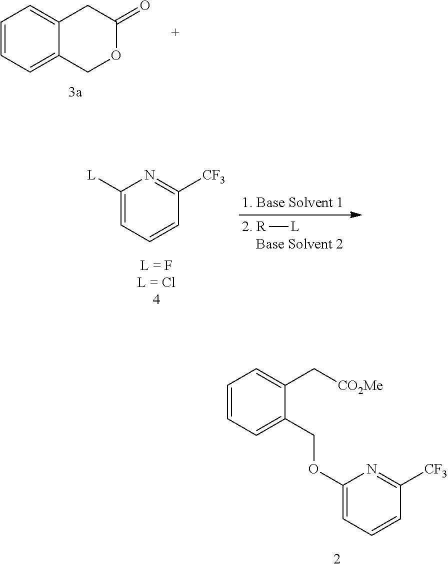

TABLE 1
Step (1)-(2)

| | | | |
|---|---|---|---|
| | | | 3 |
| MOH | Solvent 1/2 | R-L | (% Yield) |

4: L = F

| | | | |
|---|---|---|---|
| NaOH | H₂O/Acetonitrile | Me₂SO₄ | 98 |
| NaOH | Acetonitrile | Me₂SO₄ | 98 |
| NaOH | DMF | Me₂SO₄ | 93 |
| LiOH | THF/H₂O | Me₂SO₄ | 98 |
| NaOH | THF/H₂O | Me₂SO₄ | 97 |
| NaOH | Toluene | Me₂SO₄ | 94 |
| NaOH | H₂O/Toluene | Me₂SO₄ | 93 |
| NaOH | Toluene | Me₂SO₄ | 90 |

4: L = Cl

| | | | |
|---|---|---|---|
| NaOH | Toluene | Me₂SO₄ | 67 |

3-Isochromanone may be treated with 2 to 2.5 equivalents of base in solvent. After mixing base and 3-isochromanone, the reaction mixture may be stirred for 30 minutes to 24 hours. Upon completion of the reaction, water—if present—may be removed under vacuum at temperatures from 25° C. to 50° C., with or without the aid of an azeotroping solvent.

Suitable bases including lithium hydroxide, sodium hydroxide, potassium hydroxide, or mixtures thereof may be employed. Suitable solvents include water, DMF, DMSO, cyclohexanone, and THF/water mixtures. Temperatures from 25° C. to 75° C. may be employed.

To the anhydrous product of the previous reaction under a nitrogen atmosphere may be added anhydrous solvent followed by dropwise addition of 1 to 1.1 equivalents of a 2-halopyridine, for example 2-chloro-6-trifluoromethylpyridine. Suitable solvents include THE, DMF, MTBE, DMSO, cyclohexanone, and toluene. The reaction mixture may be stirred for from 5 to 32 hours at from 45° C. to 90° C. Reaction temperatures from 45° C. to 55° C., from 50° C. to 60° C., from 55° C. to 65° C., from 60° C. to 70° C., from 65° C. to 75° C., from 70° C. to 80° C. from 75° C. to 85° C., and from 80° C. to 90° C. may be used. The reaction mixture may then be cooled to between 0° C. and 10° C. Temperatures from 0° C. to 5° C., from 5° C. to 10° C., and from 10° C. to 15° C. may be used. Alternatively, the intermediate carboxylate salt may be formed by adding a solution of 3-isochromanone and 2-fluoro-6-trifluoromethylpyridine to a solvent/alkaline-base mixture.

A base, such as potassium carbonate, and solvent may then be added to the reaction mixture. Suitable solvents include DMF, cyclohexanone, toluene, MTBE, THF and DMSO. Base addition may be followed by dropwise addition of R-L, such as dimethyl sulfate, while maintaining a reaction temperature between 0° C. and 10° C. Temperatures from 0° C. to 5° C., from 5° C. to 10° C., 0° C. and 15° C. and from 10° C. to 15° C. may be used. The reaction mixture may then be warmed to a temperature of from 25° C. to 90° C. and stirred for a period of from 30 minutes to 4 hours to yield the product (2). Exemplary yields are as shown in Table 1.

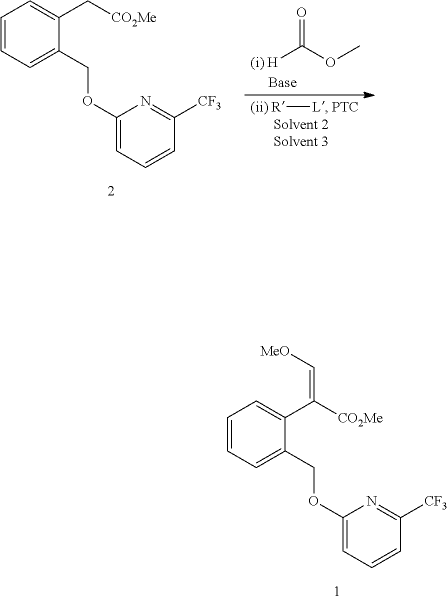

TABLE 2

| | | | | 4 |
|---|---|---|---|---|
| Base | R′-L | PTC | Solvent 2/3 | (% yield) |
| NaOMe | Me₂SO₄ | — | MeOH/Acetonitrile | 94 |
| NaOMe | Me₂SO₄ | — | DMF | 91 |
| NaOMe | Me₂SO₄ | TBAHS | MeOH/Toluene | 92 |
| NaOMe | Me₂SO₄ | TBAC | MeOH/Toluene | 88 |

TBAHS = tetrabutylammonium hydrogen sulfate;
TBAC = tetrabutylammonium chloride

An enol ether may be added to a compound of formula (II) as follows. A compound of formula (II), such as the anhydrous compound of formula (2), may be dissolved in a formate ester, such as methyl formate under a nitrogen atmosphere at a temperature of from 20° C. to 35° C., preferably from 25° C. to 30° C. followed by cooling to a temperature of from 0° C. to 5° C. A solution of sodium methoxide in methanol may be added dropwise while maintaining a temperature of from 0° C. to 5° C. The temperature may be allowed to warm to a temperature of from 20° C. to 35° C., preferably from 25° C. to 30° C., and stirred from 1 to 5 hours, preferably from 2.5 to 3.5 hours. The reaction may then be cooled to a temperature of from 0° C. to 5° C. Aqueous hydrochloric acid may then be added while maintaining the reaction temperature in a range of from 0° C. and 5° C. Acid addition may be stopped when the pH of the reaction mixture is between pH=3 and pH=4. An aldehyde/E-enol/Z-enol mixture is obtained.

A mixture of E- and Z-enols and aldehyde may be dissolved in anhydrous solvent. Suitable solvents include DMF, DMSO, acetonitrile, MTBE, and toluene. The solution may be cooled to a temperature of from 0° C. to 15° C., preferably to a temperature of from 0° C. to 10° C., followed by addition of base, such as potassium carbonate. An alkylating agent R′-L′, such as dimethyl sulfate, may then be added to the reaction mixture while maintaining a temperature of from 0° C. to 15° C., preferably from 0° C. to 10° C. Upon completion of alkylating agent addition, the reaction may be warmed to a temperature of from 20° C. to 35° C., preferably from 20° C. to 25° C. The mixture may then be stirred for an hour. A phase transfer catalyst may optionally be employed in the alkylation reaction. Suitable phase transfer catalysts include tetrabutylammonium chloride, tetrabutylammonium bromide, and tetrabutylammonium hydrogen sulfate. Excess alkylating agent may be removed with an aqueous alkaline base, such as potassium hydroxide solution, followed by extraction to isolate the product, such as a compound of formula (I). The product may be purified by recrystallization. In the case of the compound of formula (1), recrystallization may be performed with ethyl acetate/heptane.

The yield of the entire reaction sequence is in the range of from about 80% to about 90%.

### EXPERIMENTAL

In the following experimental examples, water-sensitive reagents are employed under an anhydrous nitrogen atmosphere, and anhydrous solvents are employed unless otherwise specified.

### Synthesis of Sodium 2-(2-(hydroxymethyl)phenyl)acetate

To a solution of 3-isochromanone (20.0 g) in THF (200 mL) was added NaOH (12.1 g) with stirring at RT (25° C.). After 5 minutes, water (5.5 mL, 304 mmol) was added dropwise. After stirring for 30 minutes, solvent was removed in vacuo to provide a white solid. To this solid was added THF (2×) followed by solvent removal in vacuo (2×) to yield a light grey solid, sodium 2-(2-(hydroxymethyl)phenyl)acetate.

Sodium 2-(2-(hydroxymethyl)phenyl)acetate: ¹H NMR (400 MHz, D₂O) δ 7.48-7.21 (m, 4H), 4.66 (s, 2H), 3.64 (s, 2H). LCMS: [M+1]=189. (1a) was used directly in the next step.

### Synthesis of 2-(2-(((6-(trifluoromethyl)pyridin-2-yl)oxy)methyl)phenyl)acetic Acid

To sodium 2-(2-(hydroxymethyl)phenyl)acetate was added anhydrous THF (200 mL) forming a suspension, then 2-fluoro-6-trifluoromethylpyridine (22.5 g) was added with and stirred at a temperature of from 20° C. to 30° C. The reaction mixture was heated to a temperature of from 45° C. to 55° C. for a period of from 8 to 16 hours to yield 2-(2-(((6-(trifluoromethyl)pyridin-2-yl)oxy)methyl)phenyl) acetic acid. LCMS indicated a minor amount of 2-hydroxy-6-trifluoromethylpyridine was formed.

(2-(2-(((6-(trifluoromethyl)pyridin-2-yl)oxy)methyl)phenyl) acetic acid): ¹H NMR (400 MHz, DMSO-d6) δ 12.35 (s, 1H), 7.97 (t, J=7.9 Hz, 1H), 7.49 (d, J=7.1 Hz, 2H), 7.29 (dt, J=4.8, 3.5 Hz, 3H), 7.13 (d, J=8.4 Hz, 1H), 5.38 (s, 2H), 3.73 (s, 2H). ¹³C NMR (101 MHz, DMSO-d6) δ 172.95 (s, 1C), 163.39 (s, 1C), 144.13 (q, J=34.1 Hz, 1C), 141.40 (s, 1C), 135.40 (s, 1C), 135.00 (s, 1C), 131.45 (s, 1C), 130.32 (s, 1C), 128.80 (s, 1C), 127.35 (s, 1C), 121.84 (q, J=273.7 Hz, 1C), 115.86 (s, 1C), 114.52 (d, J=3.2 Hz, 1C), 66.34 (s, 1C), 38.55 (s, 1C). ¹⁹F NMR (376 MHz, DMSO) δ −66.90 (s, 3F). LCMS: [M+1]=312, [M+23]=334

2-hydroxy-6-trifluoromethylpyridine: ¹H NMR (400 MHz, DMSO-d6) δ 11.72 (s, 1H), 7.95-7.82 (m, 1H), 7.32 (d, J=7.3 Hz, 1H), 6.95 (d, J=8.4 Hz, 1H). ¹³C NMR (101 MHz, DMSO-d6) δ 164.47 (s, 1C), 144.41 (q, J=33.7 Hz, 1C), 141.41 (s, 1C), 121.87 (q, J=273.8 Hz, 1C), 114.70 (s, 1C), 112.54 (d, J=2.9 Hz, 1C). ¹⁹F NMR (376 MHz, DMSO) δ −66.90 (s, 3F). LCMS: [M+1]=164

### Synthesis of methyl 2-(2-(((6-(trifluoromethyl)pyridin-2-yl)oxy)methyl)phenyl)acetate

The 2-(2-(((6-(trifluoromethyl)pyridin-2-yl)oxy)methyl)phenyl) acetic acid obtained from the previous reaction was cooled to a temperature of from 0° C. to 10° C., then K₂CO₃ (18.66 g) was added in one portion. Dimethyl sulfate (14.1 mL) was added dropwise while maintaining a reaction temperature of from 0° C. to 10° C. Upon completion of the dimethyl sulfate addition, the reaction mixture was warmed to a temperature of from 20° C. to 30° C. After 2 hours, the reaction mixture was cooled to a temperature of from 0° C. to 10° C. and quenched with 200 mL 0.1 mol/L KOH, the aqueous layer was extracted with 200 mL EtOAc, the combined organic layers were washed with brine. The organic phase was dried over Na₂SO₄ and concentrated to yield 42 g of methyl 2-(2-(((6-(trifluoromethyl)pyridin-2-yl)oxy)methyl)phenyl)acetate.

Methyl 2-(2-(((6-(trifluoromethyl)pyridin-2-yl)oxy)methyl)phenyl)acetate: ¹H NMR (400 MHz, DMSO-d6) δ 7.98 (t, J=7.8 Hz, 1H), 7.59-7.43 (m, 2H), 7.38-7.24 (m, 3H), 7.11 (d, J=8.4 Hz, 1H), 5.38 (s, 2H), 3.84 (s, 2H), 3.57 (s, 3H). ¹³C NMR (101 MHz, DMSO-d6) δ 171.83 (s, 1C), 163.34 (s, 1C), 144.19 (q, J=34.2 Hz, 1C), 141.38 (s, 1C), 135.41 (s, 1C), 134.39 (s, 1C), 131.51 (s, 1C), 130.51 (s, 1C), 128.91 (s, 1C), 127.56 (s, 1C), 121.83 (q, J=273.7 Hz, 1C), 115.72 (s, 1C), 114.46 (d, J=3.2 Hz, 1C), 66.32 (s, 1C), 52.02 (s, 1C), 38.11 (s, 1C). ¹⁹F NMR (376 MHz, DMSO) δ −66.85 (s, 3F). LCMS: [M+1]=326, [M+23]=348

Anhydrous methyl 2-(2-(((6-(trifluoromethyl)pyridin-2-yl)oxy)methyl)phenyl)acetate (54 g) was combined with anhydrous methyl formate (277 g) and the mixture was cooled to a temperature of from about 10° C. to about 20° C. A solution of sodium methoxide (25 wt. % in MeOH; 273.59 g) was added over a period of from about 30 minutes to about 90 minutes. Upon completion of the addition, the mixture was warmed to a temperature of from about 20° C. to about 30° C. and stirred for a period of from about 1 to about 3 hours. At the end of this time the mixture was cooled to a temperature of from about 0° C. to about 5° C. A dilute HCl solution (2.5 equiv.) was added to the mixture, while maintaining the temperature within a range of from about 0° C. to about 15.0° C., until a pH of from about 3.5 to about 5 was observed. The mixture was extracted with MTBE and the solvent was removed under vacuum to provide the product methyl 2-(2-(((6-(trifluoromethyl)pyridin-2-yl)oxy)methyl)phenyl)acetate as a mixture of E and Z isomers as well as aldehyde.

Enols: ¹H NMR (400 MHz, DMSO) δ 11.02 (s, 1H), 7.95 (t, J=7.7 Hz, 1H), 7.84 (s, 1H), 7.55-7.50 (m, 1H), 7.46 (d, J=7.3 Hz, 1H), 7.33-7.29 (m, 2H), 7.16-7.11 (m, 1H), 7.08 (d, J=8.4 Hz, 1H), 5.23 (s, 2H), 3.54 (s, 3H). ¹⁹F NMR (376 MHz, DMSO) δ −66.90 (s, 3F). LCMS [M+1]=354, [M+23]=376

Aldehyde: ¹H NMR (400 MHz, DMSO) δ 9.14 (s, 1H), 8.50 (s, 1H), 7.93 (t, J=7.9 Hz, 1H), 7.44 (d, J=7.3 Hz, 1H), 7.32 (d, J=7.3 Hz, 1H), 7.15-7.07 (m, 3H), 7.05-6.99 (m, 1H), 5.20 (s, 2H), 3.39 (d, J=4.0 Hz, 3H). ¹⁹F NMR (376 MHz, DMSO) δ −67.01 (s, 3F). LCMS [M+1]=354, [M+23]=376

### Synthesis of (E)-Methyl 3-methoxy-2-(2-(((6-(trifluoromethyl)pyridin-2-yl)oxy)methyl)phenyl)acrylate

The anhydrous products of the previous reaction, including 3-hydroxy-2-(2-(((6-(trifluoromethyl)pyridin-2-yl)oxy)methyl)phenyl)acrylate, (~80.0 g) were dissolved in acetonitrile (392 g). Potassium carbonate (57.0 g) was added and the mixture was stirred and warmed to a temperature of from about 45° C. to about 55° C. Dimethyl sulfate 23 g was added and the mixture was stirred for a period of from about 2 to about 5 hours. Water (92.0 g) was added and the mixture was stirred for a period of from about 30 minutes to about 2 hours. After analysis indicated completion of the reaction the mixture was cooled to a temperature of from about 15° C. to about 25° C. The reaction mixture was extracted with MTBE and the solvent was removed in vacuo. The crude product was dried via azeotropic distillation with acetonitrile to yield (E)-Methyl 3-methoxy-2-(2-(((6-(trifluoromethyl)pyridin-2-yl)oxy)methyl)phenyl)acrylate, a yellow oil.

### Crystallization of (E)-Methyl 3-methoxy-2-(2-(((6-(trifluoromethyl)pyridin-2-yl)oxy)methyl)phenyl)acrylate

To crude (E)-Methyl 3-methoxy-2-(2-(((6-(trifluoromethyl)pyridin-2-yl)oxy)methyl)phenyl)acrylate (7.24 g) was added EtOAc (3.6 mL). Then n-heptane (50 mL) was added dropwise with stirring. A white solid gradually precipitated. Upon completion of n-heptane addition, the suspension was cooled to a temperature of from 0° C. to 5° C. After stirring for a time of from 30 minutes to two hours, the suspension was filtered and the solid was washed with n-heptane (10 ml×3). (E)-Methyl 3-methoxy-2-(2-(((6-(trifluoromethyl)pyridin-2-yl)oxy)methyl)phenyl)acrylate (5.33 g) was obtained. (purity 98% in HPLC)

(E)-Methyl 3-methoxy-2-(2-(((6-(trifluoromethyl)pyridin-2-yl)oxy)methyl)phenyl)acrylate: 1H NMR (400 MHz, DMSO) & 7.96 (t, J=7.9 Hz, 1H), 7.61 (s, 1H), 7.55-7.49 (m, 1H), 7.48 (d, J=7.3 Hz, 1H), 7.36-7.28 (m, 2H), 7.18-7.10 (m, 1H), 7.08 (d, J=8.4 Hz, 1H), 5.23 (s, 2H), 3.79 (s, 3H), 3.57 (s, 3H). ¹⁹F NMR (376 MHz, DMSO) δ −66.92 (s, 3F). LCMS [M+1]=368, [M+23]=390

### Synthesis of Picoxystrobin

To a 2 L jacketed reactor, toluene (300 g, 7.26 eq), sodium methoxide (84.90 g, solid, purity: 99.2%, 3.48 eq) and methanol (29.91 g, 2.03 eq) were added at room temperature. A solution of methyl 2-(2-(((6-(trifluoromethyl)pyridin-2-yl)oxy)methyl)phenyl)acetate (~384 g, 38.10% in toluene, 1 equiv.) and methyl formate (143.23 g, 97.2% purity, 5.17 eq) was added over a period of 1 hr 45 minutes at room temperature. The reaction mixture was stirred at 25° C. and reaction progress was monitored by HPLC. The reaction mixture was cooled to 0° C. and 125 g of saturated HCl solution (44.9%) in methanol was added. The reaction mixture was stirred for 30 minutes at 15° C. Deionized water (510.26 g, 63.19 eq) was added. The mixture was stirred for 30 min. at 15° C. and the mixture was allowed to settle for 30 minutes. The organic and aqueous phases were separated. The organic layer (739 g) contained the formylated intermediate methyl 2-(2-(((6-(trifluoromethyl)pyridin-2-yl)oxy)methyl)phenyl)acetate as a mixture of E and Z enols and aldehyde. The mixture was used in subsequent steps.

### In Situ Phase Transfer Catalyst Formation

A mixture of methyl 2-(2-(((6-(trifluoromethyl)pyridin-2-yl)oxy)methyl)phenyl)acetate (E and Z isomers and aldehyde ~48 g dissolved in toluene; 246 g total) was added to a 1 L jacketed reactor equipped with an overhead stirrer, reflux condenser, and nitrogen inlet. Tributylamine (2.77 g 0.015 mol; 0.1 mol eq relative to methyl 2-(2-(((6-(trifluoromethyl)pyridin-2-yl)oxy)methyl)phenyl)acetate) and 26.26 g of DMS (Purity: 99.7%, 1.39 eq with respect to methyl 2-(2-(((6-(trifluoromethyl)pyridin-2-yl)oxy)methyl)phenyl)acetate) were added simultaneously over 15 min at 0-5° C.

To the above reaction mixture was added a 10 wt % solution of KOH in water (210.92 g, 95% purity, 2.37 eq relative to methyl 2-(2-(((6-(trifluoromethyl)pyridin-2-yl)oxy)methyl)phenyl)acetate) at 2-6° C. over 60 min. The reaction mixture was heated to 25° C. and stirred for 2 hours. The reaction progress and the DMS content were monitored by HPLC and GC analysis, respectively. Upon completion, the reaction mixture was allowed to settle for 15 min and the phases were separated. The aqueous layer was further extracted with toluene (84.11 g, 6.10 eq) at 25° C. The combined organic layers (314 g total) were distilled to remove solvent and crystallized to yield 49.9 g of picoxystrobin (90% yield, 98.3 wt %).

### Analytical Methods

LCMS was performed with a HALO 90 Å C18, 2.7 μm, 4.6*30 mm column, at a temperature of 45° C. Two eluent phases (A, B) were employed: A: 99.5% water, 0.05% formic acid; B: 99.5% acetonitrile, 0.05% formic acid. The elution profile is shown in the Table below:

TABLE 3
LCMS Elution method

| Time | A % | B % | Flow rate |
|---|---|---|---|
| 0.00 min | 95% | 5% | 1.8 ml/min |
| 0.80 min | 5% | 95% | 1.8 ml/min |
| 1.60 min | 5% | 95% | 1.8 ml/min |
| 1.61 min | 95% | 5% | 1.8 ml/min |
| 2.00 min | 95% | 5% | 1.8 ml/min |

NMR analysis was performed as follows:

¹H NMR: 400 MHz, DMSO-d6; ¹³C NMR: 101 MHz, DMSO-d6; ¹⁹F NMR: 376 MHz, DMSO-d6

1. A process for preparing a compound of formula (II) 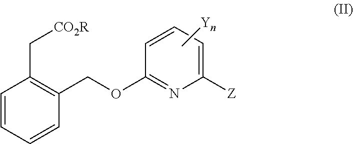
wherein Z is halo, haloalkyl, haloalkoxy, phenyl, phenoxy, amino, alkylamino, dialkylamino, acylamino, cyano, carboxy, alkoxycarbonyl, or alkylcarbonyloxy; and Yₙ wherein n=0, 1, 2, or 3 is independently halo, haloalkyl, haloalkoxy, phenyl, phenoxy, amino, alkylamino, dialkylamino, acylamino, cyano, carboxy, alkoxycarbonyl, or alkylcarbonyloxy; and R is C₁ to C₄, branched or unbranched, alkyl, alkenyl, alkynyl, haloalkyl, haloalkenyl, or haloalkynyl which comprises:
a) reacting a 2-fluoropyridine of formula (IV) in a solvent; 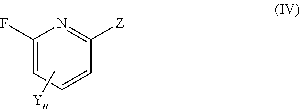
with a compound of formula (III) wherein M is lithium, sodium, potassium, or any other metal suitable for making alkoxides and carboxylates; 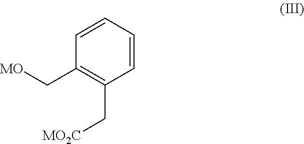
to obtain a compound of formula (V); and 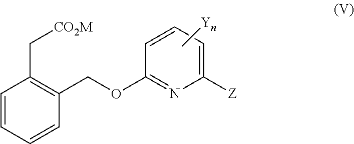
b) reacting the compound of formula (V) with a compound of formula R-L, wherein L is fluoro, chloro, bromo, iodo, sulfate, fluorosulfonate, alkylsulfonate, phenylsulfonate, substituted phenylsulfonate, or the equivalent, to obtain a compound of formula (II).

2. The process of claim 1 wherein n is 0, Z is CF₃, and R is CH₃.

3. The process of claim 2 wherein M is Na and R-L is dimethyl sulfate.

4. The process of claim 3 wherein the steps are performed with a solvent selected from the group consisting of water, acetonitrile, DMF, THF, toluene, and mixtures thereof.

5. The process of claim 4 wherein the solvent is toluene.

6. The process of claim 4 wherein 3-isochromanone is reacted with sodium hydroxide in a solvent to produce a compound of formula (III) to which is then added a solution of 2-fluoro-6-trifluoromethylpyridine.

7. The process of claim 4 wherein a solution of 3-isochromanone and 2-trifluoro-6-trifluoromethylpyridine are added to sodium hydroxide in a solvent.

8. The process of claim 1 wherein the steps are performed in one pot.

9. The process of claim 1 wherein the steps are performed in a continuous flow system.

10. A process for preparing a compound of structure (I) wherein Z is halo, haloalkyl, haloalkoxy, phenyl, phenoxy, amino, alkylamino, dialkylamino, acylamino, cyano, carboxy, alkoxycarbonyl, or alkylcarbonyloxy; and wherein Yₙ is independently halo, haloalkyl, haloalkoxy, phenyl, phenoxy, amino, alkylamino, dialkylamino, acylamino, cyano, carboxy, alkoxycarbonyl, or alkylcarbonyloxy and n=0 to 3; and wherein R and R′ are the same or different and selected from C₁ to C₄, branched or unbranched, alkyl, alkenyl, alkynyl, haloalkyl, haloalkenyl, or haloalkynyl; which comprises: 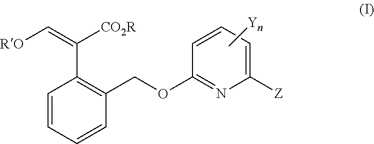
a) reacting a 2-fluoropyridine of formula (IV) in a solvent; 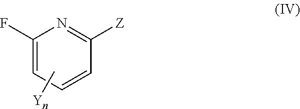
with a compound of formula (III) wherein M is lithium, sodium, potassium, or any other metal suitable for making alkoxides and carboxylates; 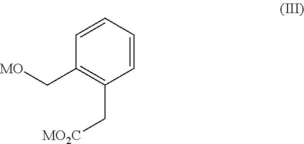
to obtain a compound of formula (V); and 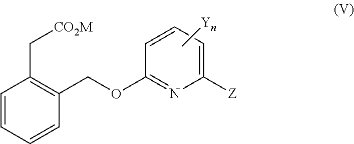
b) alkylating a compound of formula (V) with a compound of formula R-L, wherein L is fluoro, chloro, bromo, iodo, sulfate, fluorosulfonate, alkylsulfonate, phenylsulfonate, substituted phenylsulfonate or the equivalent; to obtain a compound of formula (II); and 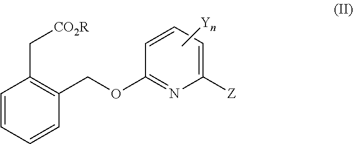
c) reacting a compound of formula (II) with an alkyl formate to form a mixture of compounds of formula (IX); and 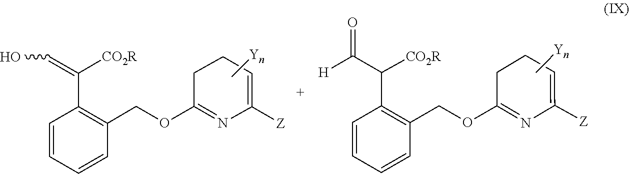
d) alkylating the mixture of compounds of formula (IX) with a compound of formula R′-L′ wherein and L′ is fluoro, chloro, bromo, iodo, sulfate, fluorosulfonate, alkylsulfonate, phenylsulfonate, substituted phenylsulfonate or the equivalent; to form a compound of formula (I).

11. The process of claim 10 wherein n is 0, Z is CF₃, R is CH₃, and R′ is CH₃.

12. The process of claim 11 wherein M is Na, R′-L′ is dimethyl sulfate, and R-L is dimethyl sulfate.

13. The process of claim 12 wherein the steps are performed with a solvent selected from the group consisting of water, acetonitrile, DMF, THF, toluene, and mixtures thereof.

14. The process of claim 13 wherein the solvent is toluene.

15. The process of claim 13 wherein 3-isochromanone is reacted with sodium hydroxide in a solvent to produce a compound of formula (III) to which is added a solution of 2-fluoro-6-trifluoromethylpyridine.

16. The process of claim 13 wherein a solution of 3-isochromanone and 2-trifluoro-6-trifluoromethylpyridine are added to sodium hydroxide in a solvent.

17. The process of claim 10 wherein the steps are performed in one pot.

18. The process of claim 10 wherein the steps are performed in a continuous flow system.

19. The process of claim 13 wherein the final methylation reaction employs a phase transfer catalyst.

20. The process of claim 13 wherein the phase transfer catalyst is formed in situ.

21. The process of claim 13 wherein the phase transfer catalyst is selected from the group consisting of tetrabutylammonium hydroxide, tetrabutylammonium hydrogen sulfate, tetrabutylammonium chloride, and tetrabutylammonium bromide.

22. The process of claim 13 wherein the final product is purified by recrystallization.
